(19) United States
(12) Patent Application Publication
(10) Pub. No.: US 2015/0290386 A1
(43) Pub. Date: Oct. 15, 2015

(54) EXTRACORPOREAL AUTOIMMUNE SOLUTION THERAPY (EAST)

(71) Applicants: Xianfeng Frank Zhao, San Diego, CA (US); Merry Y. Zhao, San Diego, CA (US)

(72) Inventors: XIANFENG FRANK ZHAO, San Diego, CA (US); Merry Y. Zhao, San Diego, CA (US)

(21) Appl. No.: 14/250,359

(22) Filed: Apr. 10, 2014

(51) Int. Cl. A61M 1/36

(52) U.S. Cl. CPC A61M 1/3687

(57) ABSTRACT

A therapeutic procedure, Extracorporeal Autoimmune Solution Therapy (EAST), is invented for eliminating pathogenic or pathologic antibodies and immune complexes via affinity column capture, specific enzyme cleavage, and specific immune suppression/immune modulation via reinfusion of the autoantibody Fab, F(ab′)₂ or Fc fragments back into the patients with a variety of acute or chronic autoimmune, inflammatory, and related other diseases.

### FIELD OF THE INVENTION

The present invention generally relates to the fields of immunology and antibody-mediated inflammatory diseases, degenerative diseases, malignancies, and autoimmune diseases.

### BACKGROUND

Autoimmune diseases are a group of the most common human diseases that are often caused by circulating autoantibodies secondary to both benign and malignant conditions. The mainstay therapy is currently still immunosuppression, which frequently causes serious infections (1). Systems have been invented to eliminate the harmful autoantibodies during autoimmune crisis. The commercially available systems include plasmapheresis and immunoadsorption (2-7). Several systems (Prosorba, Immunosorba & Globaffin, etc.) have been approved by FDA for the treatment of autoimmune diseases (8, 9). Since removal of the autoantibodies frequently lead to the rebound autoantibody production (10), none of those systems work well without simultaneous administration of immunosuppressants, which expose patients to undesirable adverse effects, such as rare infections that in some cases are life-threatening (11, 12). Because of its high cost, high complexity, short lived effects, and frequent adverse reactions, plasmapheresis and immunoadsorption are not routinely used to treat autoimmune diseases. However, no better solution is currently available to treat the autoimmune diseases. Recent studies showed that antibody Fab fragments could inhibit immune response in animal models (13). In the present invention, we combine the autoantibody removal and the specific feedback immunosuppression by the patient's own autoantibody Fab, F(ab′)₂, and/or Fc fragments to provide an optimal therapeutic solution to autoimmune diseases and related other diseases.

### SUMMARY

The present invention solves the foregoing problems associated with the current therapies for autoimmune diseases. The current therapeutic strategies for treating autoimmune diseases include immunosuppression, immunomodulation, and plasmapharesis or immunoadsorption. The immunosuppression and immunomodulation utilize specific or nonspecific reagents to decrease the subject's overall immune responses to the antigens that elicit the immune response, with the price of compromising the immune defense and immune surveillance. The immunoadsorption involves separating blood cells from plasma through centrifugation, filtering the plasma through the immunosorbant column, recombining the blood cells and the autoantibody-depleted plasma, and infusing back to the patients (8, 9). There are two technical problems with the currently available arts. 1) Separating blood cells from plasma by centrifugation and recombining the blood cells with the treated plasma are not only costly, but also likely steps for introducing harmful microorganisms. At least one incident has prevented Cypress Bioscience, Inc. from continuing to produce Prosorba columns (14). 2) The effects of current arts are short lived, always requiring immunosuppression or immunomodulation to prevent the rebound autoantibody production (10), which often produces undesirable adverse effects.

The present invention creates an optimal solution to autoimmune diseases. This said art has several features: 1) It immobilizes the specific autoantibody binders to solid polymer matrix that create a meshwork to capture the autoantibodies while normal blood flows through the column; no separation of plasma from blood cells is required for the autoantibodies to be captured in the high affinity Capture Column. Since there is no separation of the blood cells from plasma, reconstitution of blood is not required either. 2) After ˜70% of the autoantibodies have been captured, the Capture Column with bound autoantibodies is taken off the blood flow and incubated with the Immobilized Enzymes that cleave antibodies into Fab, F(ab′)₂ and/or Fc fragments. 3) The Immobilized Enzymes are separated easily from the Fab, F(ab′)₂, and/or Fc fragments with simple filtration and/or magnet capture. 4) The Fab, F(ab′)₂ and/or Fc fragments are reinfused back to the patients to either suppress the immune response of the B lymphocytes to the antigens or cover up the antigenic epitopes to prevent them from stimulating immune responses.

The present system performs two functions at the same time: 1) removal of the harmful autoantibodies from the subject (i.e. patient), which has been proved to be effective by some previous arts; 2) specific immunosuppression/immunomodulation of the immune response of the subject by his/her own autoantibodies through a feedback suppression and self-modulation mechanism. The present system has fewer steps and thus decreases the costs as well as opportunistic infections. Due to its simplicity, low cost, optimal efficiency, and minimal adverse effects, this system intends to be routinely used for the treatment of antibody-mediated autoimmune diseases. It revolutionizes the autoimmune therapeutics.

The advantages of this invention are: 1) A simple and extracorporeal system that removes the harmful autoantibodies from the body of a patient with autoimmune diseases; 2) No separation and reconstitution of blood are required for the autoantibody capture in the affinity column; 3) One step separation of the immobilized enzymes and their cleaved Fab, F(ab′)₂, or Fc fragments; 4) Infusing the harvested specific autoantibody Fab, F(ab′)₂, and/or Fc fragments back to the patients; 5) Avoiding the potential adverse effects of immunosuppressants and immunomodulants. The invented therapy is an all-natural treatment without introducing extrinsic reagents (except for PBS or NS) into the body.

The said Capture Column (FIG. 1) for the binding of autoantibodies comprises: 1) an inlet vascular access conduit with switch (not shown) for up-taking body fluid from the subject, the body fluid comprising blood or plasma and other body fluids; 2) a column containing solid matrix crosslinked with either nonspecific (Protein A, Protein G, or Protein A/G, etc.) or specific (purified proteins, peptides, or nucleic acids, etc.) antibody binders; the meshwork or spaces created by the filling matrix allowing whole blood to pass with minimal resistance (FIG. 2); having an inlet end and an outlet end that connect with the inlet and outlet vascular access conduits; 3) an outlet vascular access conduit with a switch (not shown) and catheterized with the vasculature or body cavity of the subject.

The said Enzymes that cleave antibodies into Fab, F(ab′)₂, and Fc fragments are immobilized to the said smaller solid polymer matrix or iron beads using the currently available standard protocols. The said Immobilized Enzymes will be added to the Capture Column bound with autoantibodies (FIG. 3).

The Separation Column (FIG. 4) has an inlet, body, and an outlet. A filter membrane is fixed at the outlet (variant A) to capture the Immobilized Enzymes and to allow the cleaved Fab, F(ab′)₂, and/or Fc fragments to pass through and be collected in the Collector (FIG. 5).

The variant B Separation Column (FIG. 4) has an inlet, body, and an outlet. With or without a filter membrane at the outlet, it has magnet surrounding the column wall to capture the Enzymes immobilized to the iron beads. The cleaved Fab, F(ab′)₂, and/or Fc fragments pass through the Column freely into the Collector (FIG. 5).

All the therapeutic procedures are under sterile conditions (FIG. 6). The procedures start with catheterization of two of the patient's peripheral veins. The venous blood is pumped to the Capture Column from one vein and pumped back to the patient via the other vein. After approximately 70% of the autoantibodies have bound to the Column, the Column is removed and washed with PBS or NS at the room temperature. The Immobilized Enzymes are added to the Capture Column loaded with autoantibodies for incubation to cleave the antibodies into Fab, F(ab′)₂, and Fc fragments. After the cleavage, the autoantibody Fab, F(ab′)₂, and Fc fragments are eluted through the Separation Column, wherein the Immobilized Enzymes are captured and the Fab, F(ab′)₂, and Fc fragments are collected into the Collector. The collected Fab, F(ab′)₂, and Fc fragments are infused back to the patients.

### FIGURES

FIG. 1 is a diagram illustrating the autoantibody Capture Column.

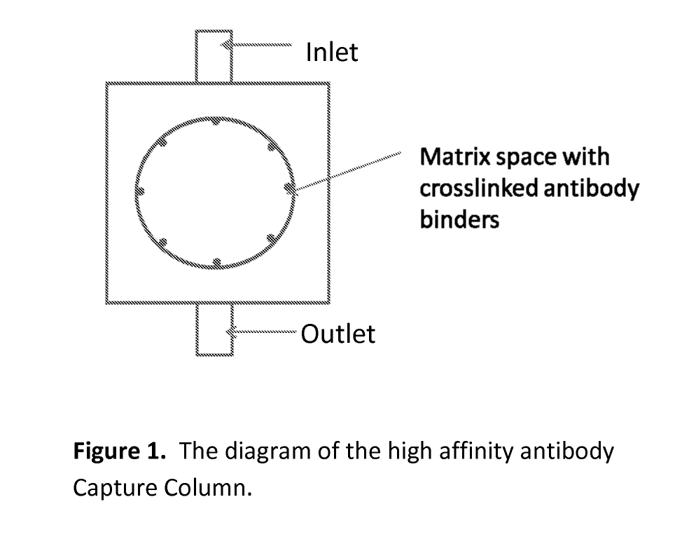

FIG. 2 is a diagram illustrating the process of autoantibody capture.

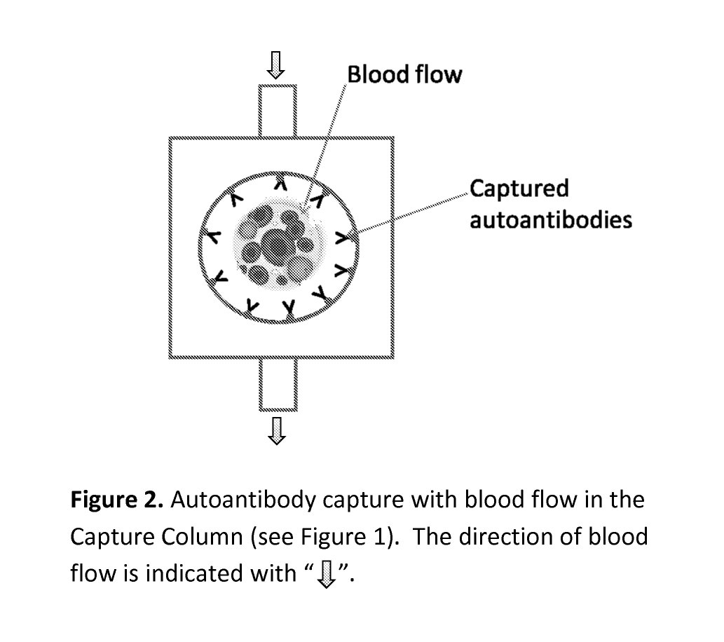

FIG. 3 is a diagram illustrating the process of autoantibody cleavage by the Immobilized Enzymes.

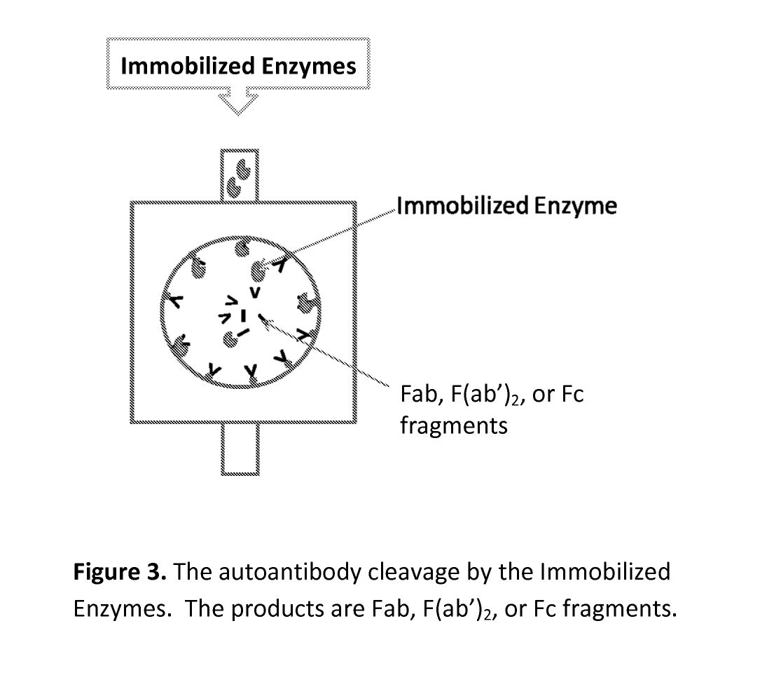

FIG. 4 is a diagram illustrating the Separation Column in work: A) Separation Column with the filter membrane; B) Separation Column with the surrounding magnet and with or without the filter membrane.

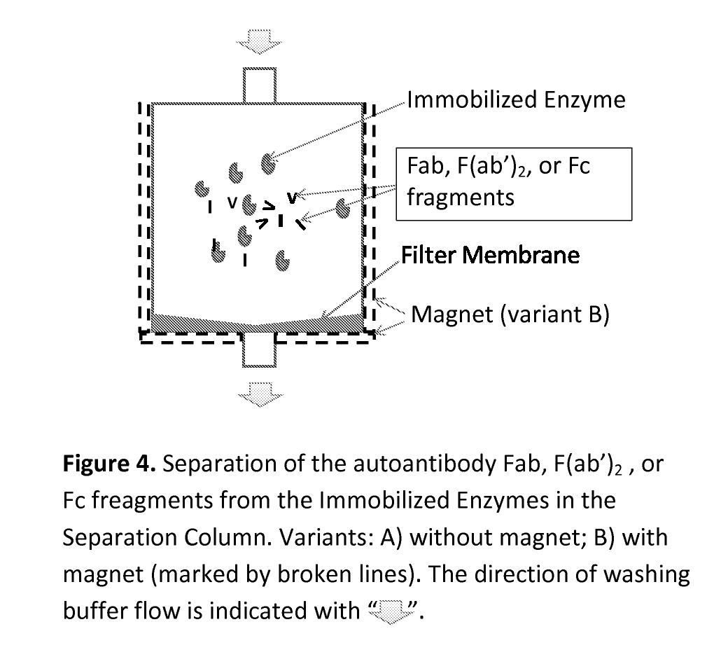

FIG. 5 is a diagram illustrating the separation of autoantibody fragments from the Immobilized Enzymes and collection of the eluted antibody fragments.

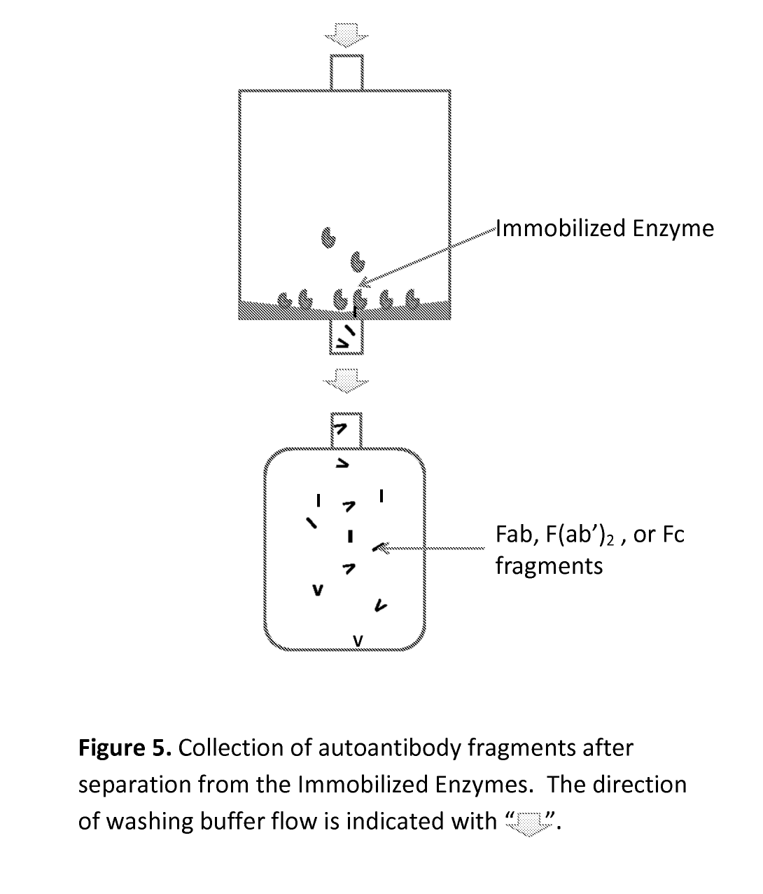

FIG. 6 is a diagram illustrating the stepwise therapeutic procedures.

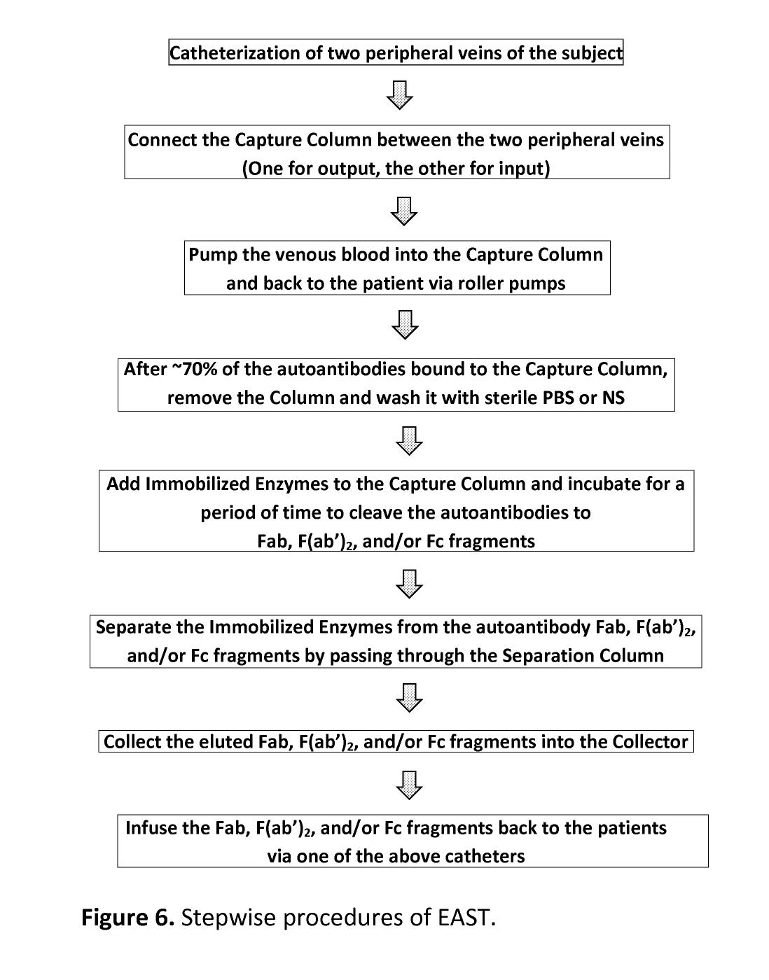

### EMBODIMENTS

Autoimmune diseases are among the most common human diseases and the 4^{th} cause of disability (15). The prevalence is approximately 5-10% of the general U.S. population and two third of the autoimmune diseases affect women (16). The world's largest pharmaceutical companies have all developed drugs for autoimmune diseases and almost all the developed drugs are either immunosuppressants or immunomodulants (17). Plasmapheresis and immunoadsorption are used in combination with immunosuppressants only for autoimmune crisis. The present invention, independent of immunosuppressant/immunomodulants, can be employed to treat both autoimmune crisis and common autoimmune diseases due to its minimal adverse effects. Only in the U.S., 23.5 million people (18) will benefit from this novel therapy.

All the Columns and related components are manufactured to the highest Healthcare Industry standard with the high quality commercially available materials (plastic columns, matrix, enzymes, proteins, peptides, etc.). For the Capture Column, the matrix will be produced first and its surfaces are immobilized with autoantibody binders, such as Protein A, Protein G, Protein A/G and/or specific antigens. The matrix will then be filled into the Column. The Enzymes used to cleave the autoantibodies are immobilized to the said smaller matrix. For the Separation Column, a membranous filter will be fixed at the outlet with or without a magnet wrapping the column. The prototypes have been tested to meet the specification and quality of design. All the components of the EAST system will be subjected to Quality Control before put to market.

The current invention combines the elimination of the circulating autoantibodies with the suppression of rebound production of those autoantibodies that is frequently seen after plasmapheresis or immunoabsorption. This system will prevent the patient from in vivo administration of the harmful immunosuppressants/immunomodulants while taking away and recycling the autoantibodies.

The said EAST procedures are designed to be performed at the Apheresis Clinics by physician assistants or registered nurses. Compared to the Prosorba and other related immunoadsorption procedures, the EAST procedures are simple, not requiring centrifuge, extra plasma, or immunosuppressants/immunomodulants. Everything comes from and goes back into the patients during the autoimmune treatment.

### Example 1

The preferred embodiment of EAST is to treat the antibody mediated autoimmune diseases using the Capture Column with nonspecific antibody binders (such as Protein A, Protein G, Protein A/G, etc.). These autoimmune diseases include rheumatoid arthritis, ITP, SLE, and some malignancies (such as chronic lymphocytic leukemia (CLL), lymphoplasmacytic lymphoma), etc. After the antibodies are captured by passing the patient's venous blood through the Capture Column, the Column will be removed and washed with PBS or NS. The bound antibodies will then be subjected to digestion by Immobilized Enzymes (such as papain, pepsin, FabRICATOR, etc.) to cleave and release the Fab and/or F(ab′)₂ fragments. The products will be eluted into the Separation Column and the Immobilized Enzymes will be separated from the Fab and/or F(ab′)₂ fragments. The Fab and/or F(ab′)₂ fragments will be infused back to the patients. Before and after each treatment, the patient's immunoglobulin levels will be measured to evaluate the effects. The eluted Fab and/or F(ab′)₂ fragments will be measured with immunoassay before each reinfusion. The goal of each treatment is to reduce the antibody levels by 70%. The patients will initially be treated every two weeks. If the drop of the antibodies is less than 50% after each treatment, the patient should be treated more frequently, such as once a week or even twice a week. If the drop is more than 70% and the antibodies remain at low level after two weeks. The treatments may be tapered to every three weeks or once a month. The patient's sign and symptoms will be closely monitored by the rheumatologists during the treatments.

### Example 2

The second preferred embodiment includes treating the autoimmune diseases with antibodies against known self-antigens, such as Myasthenia Gravis, etc. The matrix of the Capture Column will be crosslinked with specific antigens (purified proteins, peptides, nucleic acids, etc.) to capture the autoantibodies that bind those self-antigens. After the antibodies being captured, the Capture Column will be removed and washed with PBS or NS. The bound autoantibodies will then be subjected to digestion by Immobilized Enzymes (such as papain, FabRICATOR, etc.) to cleave and release the Fc fragments. The products will be eluted into the Separation Column and the Immobilized Enzymes will be separated from the Fc fragments. The Fc fragments will be infused back to the patients. Before and after each treatment, the patient's antibody levels will be measured. The eluted Fc fragments will be measured with immunoassay before each reinfusion. The goal of each treatment is to reduce the antibody levels by 70%. The patients will be treated every two weeks. If the drop of the antibodies is less than 50% after each treatment, the patient should be treated more frequently, such as once a week or even twice a week. If the drop is more than 70% and the antibody level remains low after two weeks. The treatment can be decreased to every three weeks or once a month. The patient's sign and symptoms will be closely monitored by the rheumatologists during the treatments.

The effectiveness of Fab or F(ab′)₂ fragments in Example 1 has been confirmed by several animal studies (13, 19) and the routine medical practice that using human immunoglobulin to treat ITP (20) or Rhogam to treat Rhesus disease (21), although their mode of action remains unclear (22). However, the effectiveness of Fc fragments in Example 2 has not been proven by studies or clinical practice; thus animal studies and clinical trials will be performed before its use.

### Example 3

Although it is primarily designed for treating autoimmune diseases, this therapeutic strategy will also embody eliminating any harmful molecules from the body fluids with a similar approach. Using the same strategy to capture the circulating abnormal proteins or other molecules in the human body, such as the β amyloids, ApoE, and Tau in Alzheimer disease and prion in CJ disease (mad cow disease). The Capture Column will be filled with matrix immobilized with specific antibodies against β amyloids or other circulating abnormal molecules. Those molecules will be captured when the venous blood passes through the Capture Column. The Column will be removed and treated with certain Immobilized Enzymes. The enzyme products can also be reinfused to treat diseases. This new concept, however, demands further studies.

### REFERENCES

1. Gwak G Y, Koh K C, Kim H Y. Fatal hepatic failure associated with hepatitis B virus reactivation in a hepatitis B surface antigen-negative patient with rheumatoid arthritis receiving low dose methotrexate. Clin Exp Rheumatol. 2007; 25: 888-889.
2. Davidson A and Diamond B. Autoimmune diseases. N. Engl. J. Med. 2001; 354: 340-350.
3. Felson D T, La Valley M P, Baldassare A R, et al. The Prosorba column for treatment of refractory rheumatoid arthritis: a randomized, double-blind, sham-controlled trial. Arthritis Rheum. 1999; 42: 2153-2159.
4. Tebbey P. Extracorporal immunoabsorbant treatment. US Patent 20120323158A1. Dec. 20, 2012. (http://www.google.com/patents/US20120323158)
5. Immunosorba. (http://www.fresenius.com/documents/Immunosorba_englisch.pdf)
6. Immunoabsorption—selection of literature (http://www.fmc-ag.com/files/immunoabsorption_Selected_Literature.pdf)
7. Lüking A, Scheer C, Trappe V G, Gutjahr C, Schulte K. US Patent 2009/0246203A1. Jan. 24, 2007 (http://www.google.com/patents/US20090246203?cl=en)
8. Aetna. Clinical Policy Bulletin: Extracorporeal Immunoadsorption (Prosorba column). Number: 0355 (http://www.aetna.com/cpb/medical/data/300_{—}399/0355.html).
9. Fresenius Medical Care. Immunosorba & Globaffin (http://www.fmc-ag.com/122.htm).
10. Terman D S, Garcia-Rinaldi R, Dannemann B, et al. Specific suppression of antibody rebound after extracorporeal immunoabsorption. I. Comparison of sinfle vs combination chemotherapeutic agents. Clin Exp Immunol. 1978; 34: 32-41.
11. Sakoda C1, Kusaba T, Adachi T, et al. A case of Goodpasture syndrome positive for anti-GBM antibody and MPO-ANCA complicated by a variety of serious infections. Clin Nephrol. 2011; 75: 384-388.
12. Molloy E S. PML and rheumatology: the contribution of disease and drugs. Cleve Clin J Med. 2011; 78 Suppl 2: S28-32.
13. Yoshino S, Sasahara M, Hutamekalin P, Yamaki K, Mizutani N, Kuramoto H. Suppression of antibody-mediated arthritis in mice by Fab fragments of the mediating antibodies. Br J Pharmacol. 2010; 161: 1351-1360.
14. FDA. MAUDE Adverse Event Report: Cypress Bioscience, Inc. Prosorba Column Protein A Immunoadsorption Column (http://www.accessdata.fda.gov/scripts/cdrh/cfdocs/cfmaude/detail.cfm?mdrfoi_id=260970)
15. Fact Sheet Autoimmune Disease in Women: NWHIC (http://www.rightdiagnosis.com/artic/fact_sheet_autoimmune_disease_in_women_nwhic.htm)
16. Cooper G S, Bynum M L K, Somers E C. Recent Insights in the Epidemiology of Autoimmune Diseases: Improved Prevalence Estimates and Understanding of Clustering of Diseases. J Autoimmun. 2009; 33: 197-207.
17. Autoimmune disease. Rapid progress in our understanding of immune function promises more effective treatments for autoimmune disorders. Nature Biotechnology 2000; 18, IT7-IT9.
18. Autoimmune Statistics. The American Autoimmune Related Diseases Association, Inc. (http://www.aarda.org/about-aarda/mission-statement/)
19. Grantham W G, Fitch F W. The role of antibody feedback inhibition in the regulation of the secondary antibody response after high and low dose priming. J Immunol. 1975; 114: 394-398.
20. American Society of Hematology. 2011 Clinical practice guideline on the evaluation and management of immune thrombocytopenia (ITP) (file:///C:/Users/Frank/Downloads/2011-ITP-Pocket-Guide %20(1).pdf)
21. Heyman B. Antibody feedback inhibition—a biological principle of immune regulation. Transfus Med Hemother 2005; 32: 348-354.
22. Schwab I1, Nimmerjahn F. Intravenous immunoglobulin therapy: how does IgG modulate the immune system?Nat Rev Immunol. 2013; 13: 176-189.

1. A system for treating an autoimmune, inflammatory, or related other disease of a subject (i.e. patient), comprising: a) the Capture Column to bind the autoantibodies (i.e. antibodies) while allowing the patient's whole blood or plasma pass through; b) the Immobilized Enzymes that cleave the autoantibodies into Fab, F(ab′)₂, and/or Fc fragments when being incubated with the captured autoantibodies; c) the Separation Column that retains the Immobilized Enzyme while releasing the cleaved Fab, F(ab′)₂, or Fc fragments when the elutes from the Capture Column pass through; d) the Collector to collect the eluted autoantibody Fab, F(ab′)₂, and/or Fc fragments.

2. The system of claim 1, wherein the said Capture Column contains the solid polymer matrix that is selected from the group consisting of polyester, sepharose, agarose, silica, and other polymer matrix that excludes blood cells, but does not hinder the blood flow.

3. The system of claim 2, wherein the said polymer matrix is bound with nonspecific Fc-binding partners (including but not limited to Protein A, Protein G, and Protein A/G, etc.)

4. The system of claim 2, wherein the said polymer matrix is bound with specific antibody Fab-binding partners (including specific purified proteins, peptide antigens, or nucleic acids, etc.).

5. The system of claim 1, wherein the said Immobilized Enzymes include papain, pepsin, Ficin, FabRICATOR, IdeS, and other proteinases that cleave the hinge region of the said antibodies to release Fab, F(ab′)₂, or Fc fragments.

6. The system of claim 5, wherein the said Enzymes are immobilized on the polymer matrix that are much smaller than those of claim 2 and thus could pass freely through the meshwork formed by the former polymer matrix of claim 2.

7. The system of claim 6, wherein the said polymer matrix are composed of polyester, sepharose, agarose, silica, and/or other polymers.

8. The system of claim 6, wherein the said polymer matrix are small iron beads.

9. The system of claim 1, wherein the said Separation Column has a filter membrane fixed at its outlet (variant A).

10. The system of claim 9, wherein the said filter membrane retains the Immobilized Enzymes of claim 5 and permits the released Fab, F(ab′)₂, or Fc fragments to pass through.

11. The system of claim 1, wherein the said Separation Column either with or without the said filter membrane at the outlet has surrounding magnet (variant B) to catch the Enzymes immobilized to iron beads of claim 8 and to permit the released Fab, F(ab′)₂, or Fc fragments to pass through.

12. An EAST therapeutic procedure using the said system of claim 1 for treating an autoimmune, inflammatory, neoplastic, or paraneoplastic disease of a subject, comprising predominantly four steps: 1) Capture: under sterile conditions, catheterizing a vessel of the subject, wherein the body fluid comprises whole blood, plasma or body cavity fluid; conducting the body fluid through the Capture Column; capturing the autoantibodies that pass through the Column with high affinity, immobilized, and specific autoantibody binding partners; 2) Cleavage (or Modification): removing the autoantibody-saturated Capture Column and treating the Column with Immobilized Enzymes that cleave the antibodies to release Fab, F(ab′)₂, or Fc fragments; 3) Separation: separating the cleaved antibody Fab, F(ab′)₂, or Fc fragments from the Immobilized Enzymes; 4) Reinfusion: collecting the eluted Fab, F(ab′)₂, or Fc fragments and infusing them back into the subject.

13. The method of claim 12, wherein the said autoimmune, inflammatory, neoplastic, or paraneoplastic diseases being treated are antibody-mediated, which include but are not limited to rheumatoid arthritis, idiopathic thrombocytopenia purpura (ITP), thromboangitis obliterans, Crohn disease, psoriasis, age-related macular degeneration, asthma, COPD, graft-versus-host disease, myasthenia gravis, pulmonary eosinophilia, multiple sclerosis, systemic lupus erythematosus (SLE), sepsis, paraneoplastic diseases, and malignancies, etc.

14. The method of claim 12, wherein the said disease being treated also include the diseases that are caused by self-produced antibodies (or substances) which can be captured by the Capture Column, cleaved or modified by the Immobilized Enzymes (or other catalysts), and the products of which are separated from the Immobilized Enzymes (or other catalysts) by the Separation Column, and infused back into the subjects for treatment.
